(19) United States
(12) Patent Application Publication
(10) Pub. No.: US 2007/0187899 A1
(43) Pub. Date: Aug. 16, 2007

(54) Interactive electronic display/game device that delivers aerosolized medication or other substance

(76) Inventors: Peggy Ann Bush, Aptos, CA (US); Deborah Lieberman, San Francisco, CA (US)

Correspondence Address: Peggy Bush, 112 Vista Mar, Aptos, CA, 95003, US

(21) Appl. No.: 11/283,260

(22) Filed: Feb. 14, 2006

(51) Int. Cl. G09B 23/28; A61M 11/00; A63F 9/24

(52) U.S. Cl. 273/460; 434/262; 434/322; 128/200.14

(57) ABSTRACT

This application describes an interactive electronic display/game system that delivers aerosolized medication or other substance This invention interfaces the means to deliver aerosolized product with an electronic display to encourage compliance with the correct use of the aerosolized product. The display can consist of an interactive game to reward the user for consistent and correct use of the product. The display can also provide data collected during the actual use of the product for review by the user and caregivers.

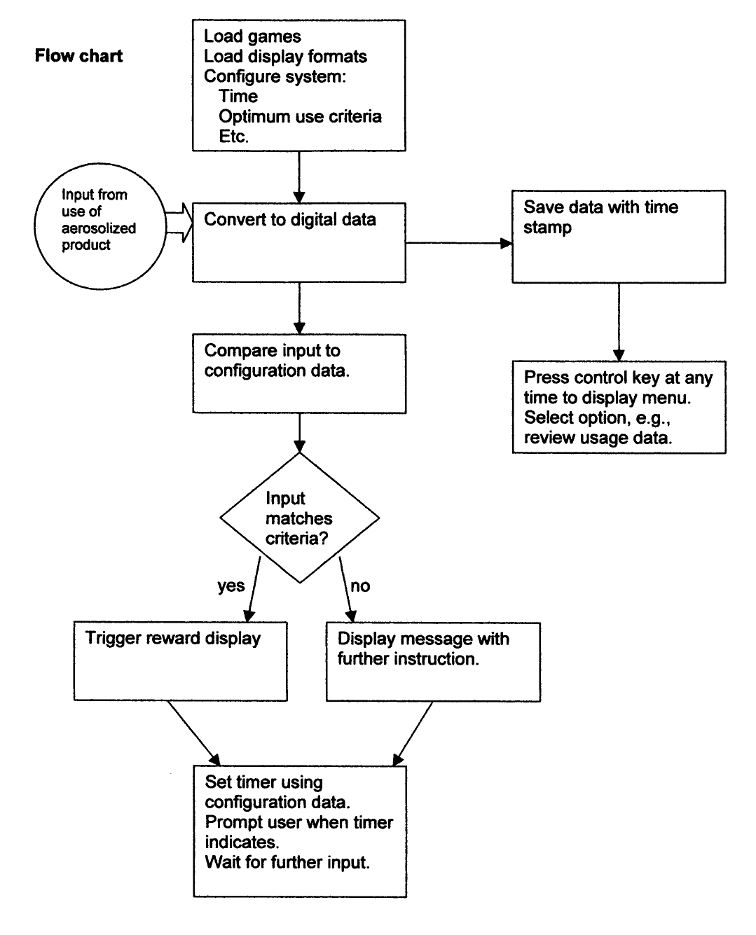

### OBJECTIVE OF THE INVENTION

This interactive real-time system delivers aerosolized medication, or other substance, and displays an interactive digital game or a passive entertainment display. Data collected about the use of the system can be viewed at any time.

This Invention Will:

Reinforce the consistent and correct use of the means to dispense aerosolized medication or other substance, by providing a programmable interface to receive input when the user uses the product and provide output to a digital game or passive entertainment.

Reward the user for consistent and correct use of medication, or other substance, and/or delivery devices (timing, frequency, method/force and/or delivery of medication or other substance) to ensure optimal efficacy.

Reward the user with a passive display reporting the user's effort and/or a multilevel digital interactive game that increases in interest and length as the user provides data consistent with programmable variables customized to reflect the desired use of the product.

The game and entertainment displayed may be adjusted for age appropriate feedback.

The system will prompt the player to use the substance delivery component at intervals appropriate to recommended regime(s). Game points will increase for consistent and correct use.

This is a portable sef-contained system that provides real time feedback on the use of the medication or other product.

The system will prevent overuse of aerosolized product (i.e. children's asthma inhalers). The programmable interface will include a time-out (or lock-out) interval to ensure the product/device is not overused. Dispensing of medication is regulated by lock-out/fail-safe features. This is particularly useful for pulmonary lung patients who often over-use inhalers causing a “rebound” effect.

The system will tutor users (particularly children) regarding the proper use of dispensing devices through sensory feedback and stimulation.

Advantages of this Invention Over Prior Art:

The self-ontained system will be used to measure the compliance of the user against defined optimal use. Consistent use of a medication, or other product, provides optimal improvement and desired outcome(s).

The system supports self-motivation for consistent use and improved medical compliance.

The system will encourage the correct and consistent use of rescue inhalers and maintenance inhalers, for example, for delivery of broncho-dialators, bronchial topical corticosteriods, oxygen, etc. The use of this system is not, however, limited to rescue inhalers and maintenance inhalers.

This invention does not simply store data and provide rewards. The invention stimulates the user with an interactive game interface and/or static display based on user efforts.

The programmable device includes a lock-out feature to prevent over-use or over-medication.

This system will prompt the user using, for example, downloadable ring tones to notify the user to when it is time to take the medication.

The Components of the System are Listed Here.

Mouthpiece and cover.

Metered medication delivery component.

Capacity to aerosolize liquid or powder.

Chamber to hold substance to be delivered.

A display screen to display encouraging entertainment or static data based on the input.

Audio speakers.

An programable interface to accept, process and record data.

Interchangeable game cartridges.

Internal memory, batteries (conventional, rechargeable or solar).

A USB, or other wired or non-wired port to download data to another device.

Keyboard, joystick and/or other input devices.

A case to carry and protect the system.

The system will be self-contained. It may also plug-in to separate components not listed here.

### HOW DOES THIS INVENTION ACHIEVE ITS RESULTS?

This invention provides an interface for kinesthetic input from a substance delivery device to an interactive real-time electronic digital game. It may also take the form of a self-contained medication dispensing/game console combination unit. Playing digital games provides motivation to use the substance delivery device consistently and correctly, thus increasing its therapeutic value.

The display may be adjusted for age appropriate feedback. The display may be interactive and competitive or may provide passive, encouraging entertainment based on user—customizable program variables. The system is small, self contained and highly portable. It may be adapted to interface with larger and sturdier equipment for institutional hospital/clinic/ambulance therapeutic use.

The long-term data storage encourages the user to perform at a personal best and rewards long-term compliance. The system is programmed to prompt the user to use the device at defined intervals and is programmed to sense proper use.

For example, aerosolized medication may be prescribed to be used every four hours when awake. Further, the user should inhale once, wait ten seconds and use the inhaler a second time. The system will be programmed to digitize the proper use and increase the level of feedback accordingly. It will also cue to improper use, specifically, tutoring for proper use of inhalers for young children, etc. The system discourages improper use and over-use with lock-out features.

The player uses the system for home health, medical or sport purposes. The action of the player is monitored by a programmable electronic interface. The interface digitizes the user effort. A program responds to the digital data. When the player completes the programmed regime, a multi-level electronic game is displayed to provide positive immediate feedback for the user's effort. The user is motivated to use the substance delivery device consistently.

Consistent compliance over time will be recorded and reflected in the level of the game display and be available for third-parties such as parents and medical personnel.

The user is able to store points earned and delay playing the game until a time of their choosing.

The system may be used to deliver products for home health, grooming, medical or sport purposes.

Alternate Ways that the Invention Can Achieve its Result

The programmable interface and display device is adaptable to any size or shape aerosolized delivery product or may be configured to include the capacity to aerosolize a product or to contain and deliver a substance that does not require aersolization.

The programmable interface and display device may be adapted to a nebulizer.

The programmable interface and display device may be used for delivery of any aerosolized medication, for example, vaccines, hormones, antibiotics, insulin, or vitamin supplements.

The programmable interface and display device may be used to deliver a preprogrammed dose of oxygen.

The programmable interface and display device may be adapted to deliver a preprogrammed dose of ophthalmologic treatment.

The programmable interface and display device may be adapted to be used to deliver breath freshener, aerosolized sore throat remedies or personal hygiene products.

The actual games/displays programmed for the system can be interchangeable. Customizable parameters can be available.

The programmable interface with the means to deliver aerosolized product could be sold as a stand-alone attachment with programmed cartridges to enhance an existing digital game device.

The programmable interface and display device could be a stand-alone attachment to existing medical equipment, i.e. nebulizers, portable and non-portable inhalers, oxygen equipment, etc.

The system could be adapted to monitor and reward use of other health products, for example, home dialysis units or medications delivered by syringe.

I claim:

1. A portable and/or hand-held means to monitor and deliver aerosolized medical and/or health related products that promotes use consistent with optimum efficacy by providing positive feedback, comprised of:
a means for delivery of aerosolized product
a device for digital display
programmable interface with memory option between said means for the delivery of aerosolized product and said device for digital display that provides a method to analyze various types of kinesthetic input, processing same and producing digital output in various forms, including but not limited to electronic games and/or video/audio display, the purposes of such output is to effectuate and reinforce execution of and /or compliance with varying medical and/or health related regimens
